(19) United States
(12) Patent Application Publication
(10) Pub. No.: US 2023/0293226 A1
(43) Pub. Date: Sep. 21, 2023

(54) GAS PASSAGE ADJUSTMENT MECHANISM, SMOKE EVACUATION SYSTEM, AND SURGICAL SYSTEM

(71) Applicant: KYOCERA Corporation, Kyoto-shi, Kyoto (JP)

(72) Inventor: Hiroaki YOSHIKAWA, Ritto-shi, Shiga (JP)

(21) Appl. No.: 18/017,299

(22) PCT Filed: Aug. 2, 2021
(86) PCT No.: PCT/JP2021/028546

§ 371 Date: Jan. 20, 2023

(30) Foreign Application Priority Data
Aug. 7, 2020 (JP) 2020-135149

(51) Int. Cl. A61B 18/14

(52) U.S. Cl. CPC A61B 18/1482; A61B 2018/00595; A61B 2218/008

(57) ABSTRACT

To provide a gas passage adjustment mechanism that can be reduced in size. A smoke evacuator 1 (gas passage adjustment mechanism) includes a pressing member (12), a solenoid (13), a tube holder (14), a first protruding member (15), and at least one second protruding member (16). The first protruding member (15) is provided at a position facing a pressing position (P) where the pressing member (12) presses the tube (11), and protrudes from the tube holder (14) in a direction toward the pressing member (12). The at least one second protruding member (16) is disposed at an upstream position and a downstream position of the passage with respect to the pressing position (P), and protrudes toward a side of the tube (11).

### TECHNICAL FIELD

An invention according to the present disclosure relates to a gas passage adjustment mechanism and a surgical system in a smoke evacuation system for surgical smoke.

### BACKGROUND OF INVENTION

In a laparoscopic surgery with a medical cautery instrument, a suction device may suck surgical smoke generated in the abdominal cavity of a patient and may remove the surgical smoke from the abdominal cavity. A known smoke evacuator discharges the surgical smoke to the outside of the body.

### SUMMARY

A gas passage adjustment mechanism according to an aspect of the present disclosure includes a pressing member, a solenoid, a tube holder, a first protruding member, and at least one second protruding member.

The pressing member presses a tube including a passage for gas. The solenoid moves the pressing member, and thereby opens and closes the passage. The tube holder holds the tube from a side opposite to the pressing member. The first protruding member is provided at a position facing a pressing position at which the pressing member presses the tube. The first protruding member protrudes from the tube holder in a direction toward the pressing member. The second protruding member is disposed at an upstream position or a downstream position of the passage with respect to the pressing position, and protrudes toward a side of the tube.

A gas passage adjustment mechanism according to an aspect of the present disclosure includes a pressing member configured to press a tube including a passage for gas, a solenoid configured to move the pressing member and configured to open and close the passage through the movement, a tube holder including a clamping portion configured to clamp a part of the tube from a side opposite to the pressing member, and a first protruding member provided at a position facing a pressing position at which the pressing member presses the tube, the first protruding member protruding from the tube holder in a direction toward the pressing member.

### BRIEF DESCRIPTION OF THE DRAWINGS

FIG. 1 is a schematic view illustrating a surgical system according to an aspect of the present disclosure.

FIG. 2 is a perspective view illustrating a main configuration of a smoke evacuator according to the aspect of the present disclosure.

FIG. 3 is a schematic cross-sectional view of the smoke evacuator according to the aspect of the present disclosure.

FIG. 4 is a schematic cross-sectional view of the smoke evacuator according to the aspect of the present disclosure.

FIG. 5 is a perspective view illustrating an example of a configuration of a tube holder according to the aspect of the present disclosure.

FIG. 6 is a perspective view illustrating an example of a configuration of a second protruding member according to the aspect of the present disclosure.

FIG. 7 is a schematic cross-sectional view of a smoke evacuator according to another aspect of the present disclosure and a perspective view illustrating an example of a tube holder.

FIG. 8 is a schematic diagram illustrating an example of a configuration of a surgical system according to an aspect of the present disclosure.

### DESCRIPTION OF EMBODIMENTS

### First Embodiment

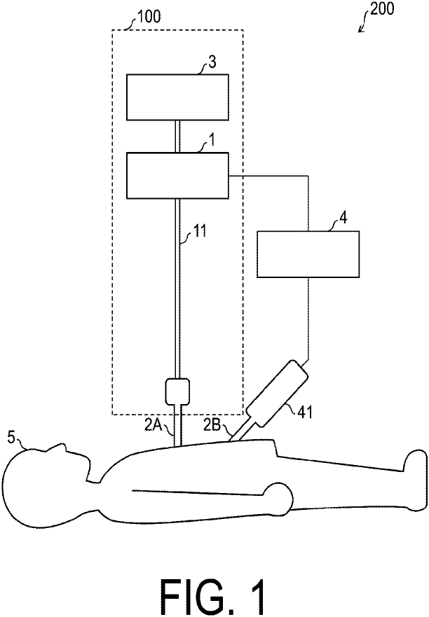

An aspect of the present disclosure will be described with reference to FIG. 1 to FIG. 7.

### Surgical System Overview

A surgical system 200 using a smoke evacuator 1 according to an aspect of the present disclosure will be described using FIG. 1. FIG. 1 is a schematic view illustrating the surgical system 200 including a smoke evacuation system 100 including a smoke evacuator 1 (gas passage adjustment mechanism) according to an aspect of the present disclosure.

In general, in laparoscopic surgery, surgical smoke may be generated in the abdominal cavity of a patient 5 due to the abdominal cavity being filled with a gas containing nitrogen or the like to maintain the air pressure in the abdominal cavity and secure a surgical field as well as due to cauterization or the like of body tissue by a surgical cautery instrument. The retention of the surgical smoke in the abdominal cavity of the patient 5 may lead to obstruction of the view of the surgical field. For this reason, the surgical smoke is preferably discharged from the abdominal cavity.

The surgical system 200 is a system for performing endoscopic surgery on the patient 5. The surgical system 200 includes a smoke evacuation system 100 and a cautery device 4. The smoke evacuation system 100 is a system for discharging the surgical smoke retained in the abdominal cavity of the patient 5. The smoke evacuation system 100 includes a trocar 2A, a tube 11, a smoke evacuator 1, and a suction device 3. In this specification, gas containing surgical smoke is simply referred to as gas.

The trocar 2A is a medical instrument including a tubular member. The medical instrument is punctured into the abdominal region of the patient 5, secures a path between the abdominal cavity of the patient 5 and the outside of the body of the patient 5, and functions as a guide tube. In particular, in the present embodiment, the trocar 2A is retained in the body wall to secure a path for smoke evacuation. The trocar 2A is connected to the tube 11 to form a part of a passage for gas from the abdominal cavity of the patient 5 to the outside of the patient 5.

The tube 11 is a tube made of an elastically deformable material. The tube 11 may be a tube to be generally used in medical practice. As the tube 11, for example, a silicon tube having an inner diameter of 5 mm is used, but the material and tube diameter of the tube 11 are not particularly limited. A first end of the tube 11 is connected to the trocar 2A and a second end is connected to the suction device 3. In other words, the tube 11 includes a passage for gas from the trocar 2A to the suction device 3.

The smoke evacuator 1 (gas passage adjustment mechanism) is a device disposed in a part of the passage for gas between the trocar 2A and the suction device 3. The device opens and closes the passage for gas by pressing the tube 11. The smoke evacuator 1 will be described in detail below.

The suction device 3 is a device that sucks gas in the abdominal cavity of the patient 5. The suction device 3 may be a device having a suction function that is generally used in medical practice. As the suction device 3, for example, medical gas piping that is connected to a suction facility by connecting the tube 11 to a pipe terminal installed on a wall surface of an operating room may be used.

The cautery device 4 is a medical cautery device including a cautery instrument 41 (surgical cautery instrument). The cautery instrument 41 is used for endoscopic surgery. Examples of the cautery instrument 41 include an ultrasonic scalpel, an electric scalpel, a laser scalpel, and cautery forceps. A treatment on the body tissues of the patient 5 with the cautery instrument 41 may be performed through, for example, the trocar 2B retained in the body wall of the patient 5. The trocar 2B may be the same member as the trocar 2A.

### Smoke Evacuator

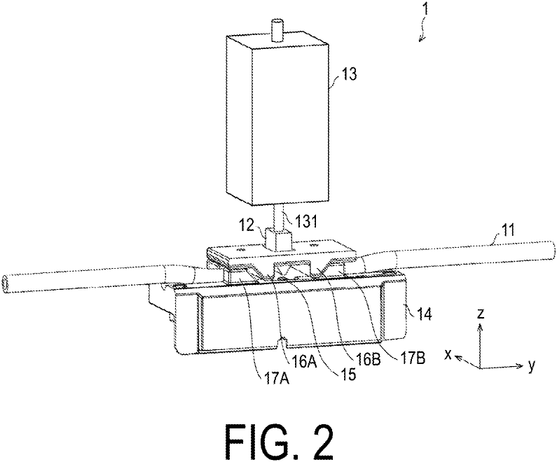

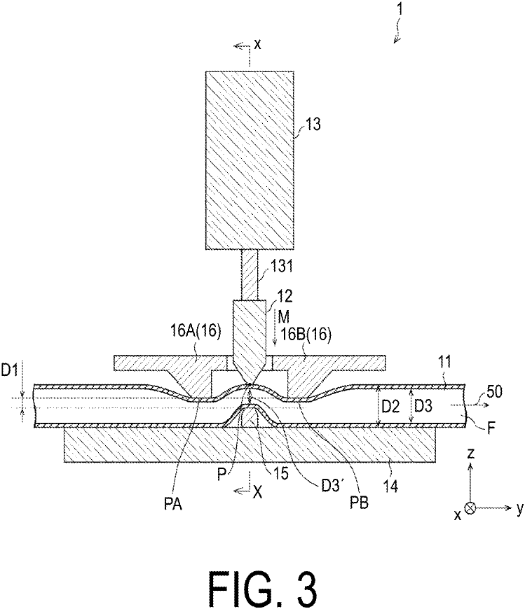

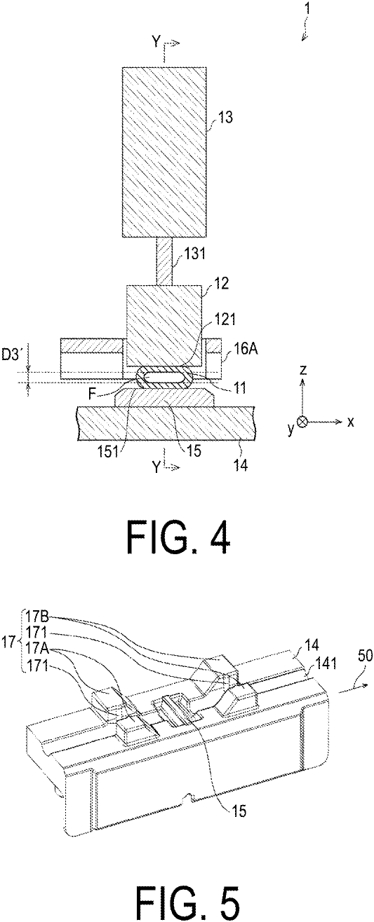

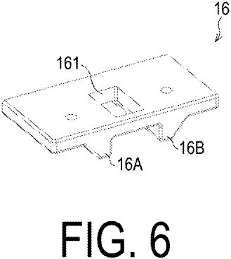

The smoke evacuator 1 according to an aspect of the present disclosure will be described using FIG. 2 to FIG. 6. FIG. 2 is a perspective view illustrating an example of a main configuration of the smoke evacuator 1. FIG. 3 is a cross-sectional view of the smoke evacuator 1 taken along a plane including the central axis of the tube 11 and the central axis of the pressing member 12. That is, FIG. 3 is a cross-sectional view of the smoke evacuator 1 taken along a section line Y-Y illustrated in FIG. 4, as viewed in a direction of the arrows. FIG. 4 is a cross-sectional view taken along a plane that is orthogonal to the cross section illustrated in FIG. 3 and that includes the central axis of the pressing member 12. That is, FIG. 4 is a cross-sectional view of the smoke evacuator 1 taken along a section line X-X illustrated in FIG. 3, as viewed in a direction of the arrows. FIG. 5 is a perspective view illustrating an example of a configuration of a tube holder 14 of the smoke evacuator 1. FIG. 6 is a perspective view illustrating an example of a configuration of a second protruding member 16. For the sake of convenience, FIG. 2 to FIG. 4 illustrate only a main portion of the smoke evacuator 1. The other elements such as a frame member for holding and fixing each of main components at a predetermined position and a housing for covering the frame and component are omitted. In FIG. 2 to FIG. 4, an axis parallel to a movement direction of the pressing member 12 is defined as a z axis, an axis parallel to an extending direction of the tube 11 is defined as a y axis, and an axis orthogonal to both the z axis and the y axis is defined as an x axis.

As illustrated in FIG. 2 to FIG. 4, the smoke evacuator 1 includes the pressing member 12, the solenoid 13, the tube holder 14, the first protruding member 15, and the second protruding member 16. In the present disclosure, two second protruding members 16 are provided. In FIG. 3, an arrow M indicates a pressing direction (movement direction) of the pressing member 12. A distance D1 indicates a distance between a tip of the first protruding member 15 and a tip of the second protruding member 16 in a direction parallel to the movement direction of the pressing member 12. A tip PA indicates a tip of a member 16A of the second protruding member 16. A tip PB indicates a tip of a member 16B of the second protruding member 16. The tip PA and the tip PB are substantially at the same distance from the tube holder 14 in a direction parallel to the z axis direction. The tip of the second protruding member 16 means the tip PA or the tip PB.

An outer diameter D2 indicates an outer diameter of the tube 11 in an unloaded state. An inner diameter D3 indicates an inner diameter of the tube 11 in an unloaded state. A distance D3′ indicates a distance between inner wall surfaces of the tube 11 at a position where the first protruding member 15 contacts the tube 11. The direction of an arrow 50 indicates a direction in which gas in a passage F flows. A pressing position P indicates a position at which the pressing member 12 presses the tube 11.

The pressing member 12 is a member that presses the tube 11. The pressing member 12 has a shape suitable for pressing the tube 11. The shape of the pressing member 12 is not particularly limited as long as the pressing member 12 can press the tube 11. For example, as illustrated in FIG. 3, a tip portion of the pressing member 12 has a tapered shape in the pressing direction. As illustrated in FIG. 4, the pressing member 12 has a shape in which a length of a ridge line of the tip portion being in contact with the tube 11 is longer than a length in a major axis direction of the pressed tube 11. That is, a contact surface 121 (see FIG. 4) of the pressing member 12 that comes into contact with the tube 11 has a thin belt-like shape extending in a direction orthogonal to the extending direction of the tube 11. Thus, when the tip portion of the pressing member 12 presses the tube 11, the tip portion of the pressing member 12 can effectively press the tube 11, and close the passage F. The pressing member 12 may have hardness capable of pressing the tube 11. As a material of the pressing member 12, for example, a metal material such as SUS or aluminum is used.

The solenoid 13 is a constituent element that moves the pressing member 12 so as to open and close the passage F. As illustrated in FIG. 2 to FIG. 4, the solenoid 13 includes a plunger 131. A front end of the plunger 131 is connected to the pressing member 12. When the plunger 131 is not energized, the tip portion of the pressing member 12 may be in contact with the tube 11 or may be positioned in close proximity to the tube 11. When excited by energization, the solenoid 13 moves the plunger 131 in the pressing direction. That is, the solenoid 13 presses the tube 11 and closes the passage F by moving the pressing member 12 in the pressing direction. That is, the distance D3′ before the pressing member 12 presses the tube 11 (in the released state of the passage F) becomes substantially zero along with the movement of the pressing member 12 in the pressing direction. Then, the passage F is closed. When the energization of the solenoid 13 is terminated, the plunger 131 moves in the direction opposite to the pressing direction. That is, the solenoid 13 opens the passage F by moving the pressing member 12 in the direction opposite to the pressing direction. In other words, when the solenoid 13 is not energized, the passage F is opened at the pressing position P. When the solenoid 13 is energized, the passage F is closed at the pressing position P.

The solenoid 13 may be a latching solenoid. The latching solenoid is a solenoid that includes a built-in permanent magnet and that is capable of maintaining the plunger at the position even during a period in which power is not supplied by attracting a built-in metal block to the built-in permanent magnet. When the solenoid 13 is a latching solenoid, and when a pulse voltage is applied, the solenoid 13 moves the plunger 131 in the pressing direction and maintains the plunger 131 at the position after the movement due to the magnetic force of the permanent magnet. While the plunger 131 is maintained at the position after the movement, the passage F is closed. When the pulse voltage is applied again, the solenoid 13 moves the plunger 131 in the direction opposite to the pressing direction. This opens the passage F. By using the latching solenoid as the solenoid 13, the plunger 131 can be maintained at the position even during a period in which power is not supplied. Thus, an energy consumption amount can be reduced.

The tube holder 14 is a member that holds the tube 11 from a side opposite to the pressing member 12. As illustrated in FIG. 5, the tube holder 14 may include a pair of holding members 17 (a holding member 17A and a holding member 17B) for holding the tube 11 at a predetermined position of the tube holder 14. The arrow 50 in FIG. 5 indicates a direction of gas flow when the tube 11 is held in the tube holder 14. One holding member 17A of the pair of holding members 17 is disposed at the upstream side of the passage F with respect to the pressing position P. The other holding member 17B is disposed at the downstream side of the passage F with respect to the pressing position P. Each of the holding member 17A and the holding member 17B includes a contact surface capable of sandwiching the tube 11 from a respective one of both sides. Each of the holding member 17A and the holding member 17B may have a protruding portion 171 at the contact surface with the tube 11 in order to improve a holding property of the tube 11. The tube holder 14 may further include a groove 141 for holding the tube 11 at a predetermined position of the tube holder 14.

With the above configuration, the tube holder 14 can hold the tube 11 at an appropriate position. This makes it possible to improve the accuracy of pressing the tube 11 with the pressing member 12.

The first protruding member 15 is a member provided at a position facing the pressing position P (see FIG. 3) at which the pressing member 12 presses the tube 11. The member protrudes from the tube holder 14 in a direction toward the pressing member 12. The first protruding member 15 is positioned at the side opposite to the pressing member 12 with the tube 11 interposed therebetween. That is, the central axis of the first protruding member 15 in the z axis direction substantially coincides with the central axis of the pressing member 12 in the z axis direction. The first protruding member 15 deforms the tube 11 in the vicinity of the pressing position P in advance so as to reduce the distance D3′.

A shape of the first protruding member 15 is not particularly limited as long as the shape can be deformed such that the distance D3′ becomes smaller. A contact surface 151 (see FIG. 4) of the first protruding member 15 that comes into contact with the tube 11 has, for example, a thin belt-like shape extending in a direction orthogonal to the extending direction of the tube 11.

The second protruding member 16 illustrated in FIG. 3 is a member that presses the tube 11 in advance such that the tube 11 comes into contact with the tube holder 14. The second protruding member 16 is a member protruding toward the tube 11 from the side opposite to the tube holder 14. That is, the second protruding member 16 protrudes in a direction in which the tube 11 is closed in the movement direction of the pressing member 12. At least one second protruding member 16 may be provided at an upstream position or a downstream position of the passage F with respect to the pressing position P. Providing the second protruding member 16 restricts the tube 11 pushed up by the first protruding member 15 from the opposite direction (anti-pressing direction) to the pressing direction from escaping in the anti-pressing direction. The distance D3′ can be further shortened by adjusting a position of the second protruding member 16 in the pressing direction. In addition, the smoke evacuator 1 may include each of a pair of second protruding members (a member 16A and a member 16B) at a respective one of an upstream position and a downstream position of the passage F with respect to the pressing position P. That is, as for the second protruding members 16, a pair of the member 16A disposed at the upstream side of the passage F and the member 16B disposed at the downstream side of the passage F may form the second protruding members 16. In this case, the second protruding members 16 are disposed so as to interpose a movement path of the pressing member 12. In other words, each of the second protruding members 16 may be disposed in a pair at a respective one of an upstream position and a downstream position in the passage F with respect to the pressing position P. With the configuration, the tube 11 can be more appropriately pressed in advance.

The pair of second protruding members 16 may be constituted by two members, or may have a shape in which the member 16A and the member 16B are integrally formed as illustrated in FIG. 6. In the case of integral formation, for example, a hole portion 161 may be formed between the member 16A and the member 16B in order to secure the movement path of the pressing member 12.

For example, as illustrated in FIG. 3, the tip of the second protruding member 16 is positioned such that the distance D1 in the z axis direction between the tip of the first protruding member 15 and the tip of the second protruding member 16 is smaller than the outer diameter D2 of the tube 11 in an unloaded state.

With the above configuration, the tube 11 is further deformed from the pressing direction by the second protruding member 16 in addition to being deformed from the anti-pressing direction by the first protruding member 15. Thus, the distance D3′ becomes shorter than the inner diameter D3 in a state before being pressed by the pressing member 12. That is, a stroke required for the pressing member 12 to close the tube 11 can be shortened in the state before being pressed by the pressing member 12. That is, a degree of decrease in the attraction force of the solenoid due to an increase in the stroke can be reduced and even a smaller solenoid than before can achieve the necessary pressing force to close the tube.

As illustrated in FIG. 4, at the pressing position P, the distance D3′ between the inner wall surfaces of the tube 11 before being pressed by the pressing member 12 may be equal to or less than 3 mm. That is, the solenoid 13 having a stroke being at least equal to or larger than 3 mm can close the tube. This can reduce the degree of decrease in the attraction force of the solenoid 13. At the pressing position P, the distance D3′ between the inner wall surfaces of the tube 11 before being pressed by the pressing member 12 may be equal to or less than 1.5 mm. In other words, when the stroke of the solenoid 13 is at least equal to or larger than 1.5 mm, the tube can be closed. As a result, the degree of decrease in the attraction force of the solenoid 13 can be reduced.

In the present embodiment, the smoke evacuator 1 includes the pressing member 12 configured to press the tube 11 forming the passage F for gas, the solenoid 13 configured to move the pressing member 12 and configured to open and close the passage F through the movement, the tube holder 14 configured to hold the tube 11 from the opposite side of the pressing member 12, the first protruding member 15 protruding from the tube holder 14 in a direction toward the pressing member 12 at a position opposite to the pressing position P at which the pressing member 12 presses the tube 11, and the pair of second protruding members 16 each of which is disposed at a respective one of an upstream position and a downstream position of the pressing position P in the passage F for gas, the pair of second protruding members 16 protruding toward a side of the tube 11.

With the above configuration, the tube 11 at the pressing position P is deformed in advance such that the distance D3′ between the inner wall surfaces of the tube 11 in the pressing direction by the pressing member 12 is reduced, and the stroke required for the pressing member 12 to close the tube can be reduced. The solenoid has a characteristic that the attraction force decreases as the stroke increases. Due to this, with the above configuration, the degree of decrease in the attraction force of the solenoid due to the increase in the stroke can be reduced. Thus, the pressing force required to close the tube even with a latching solenoid that is smaller in size than a typical latching solenoid can be achieved. Thus, a small gas passage adjustment mechanism can be implemented.

### Second Embodiment

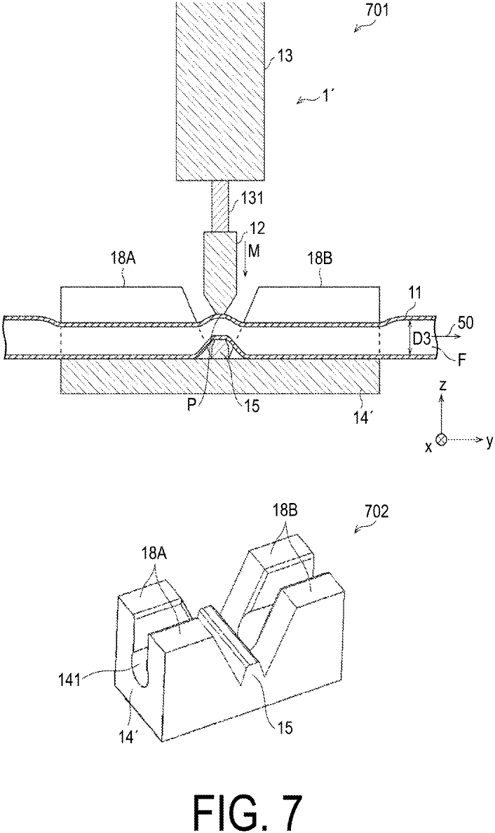

In the following, another embodiment of the present disclosure will be described with reference to FIG. 7. For convenience of description, a member having the same function as that of a member described in the embodiment described above is denoted by the same reference sign, and description thereof will not be repeated. A reference numeral sign 701 in FIG. 7 denotes a schematic cross-sectional view of the smoke evacuator 1′ according to a second embodiment. A reference numeral sign 702 in FIG. 7 denotes a perspective view illustrating an example of a tube holder 14′ according to the second embodiment. The smoke evacuator 1′ according to the second embodiment is different from the smoke evacuator 1 according to the first embodiment in that the smoke evacuator 1′ does not include the second protruding members 16 according to the first embodiment and includes the tube holder 14′ instead of the tube holder 14 according to the first embodiment.

The tube holder 14′ is a member that holds the tube 11 from the side opposite to the pressing member 12. As illustrated in FIG. 7, the tube holder 14′ includes the groove 141 for holding the tube 11 at a predetermined position in the tube holder 14′. The tube holder 14′ includes a pair of clamping portions 18 (a clamping portion 18A and a clamping portion 18B) extending from the groove 141 in the z axis direction. The pair of clamping portions 18 clamp a part of the tube 11 from the side opposite to the pressing member 12. As for the clamping portions 18, the clamping portion 18A disposed at an upstream side of the passage F and the clamping portion 18B disposed at a downstream side of the passage F form the pair of clamping portions 18. The clamping portions 18 hold the tube 11 and restrict the tube 11 pushed up by the first protruding member 15 from the opposite direction (anti-pressing direction) to the pressing direction from escaping in the anti-pressing direction. That is, the clamping portions 18 serve as both the second protruding members 16 and the holding members 17 of the smoke evacuator 1 according to the first embodiment.

With the above configuration, the smoke evacuator 1′ according to the second embodiment can provide the same effects as the smoke evacuator 1 according to the first embodiment. Thus, a small gas passage adjustment mechanism can be implemented.

### Operations of Surgical System

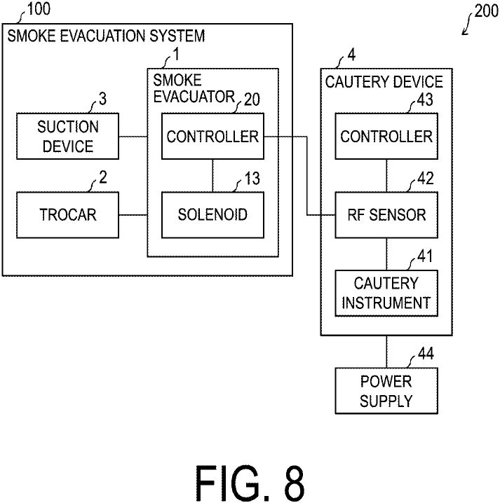

An operation of the surgical system 200 when the surgical system 200 is used to perform surgery on the patient 5 will be described with reference to FIG. 8. FIG. 8 is a schematic diagram illustrating an example of a main configuration of the surgical system 200.

The surgical system 200 includes the smoke evacuation system 100 and the cautery device 4. The cautery device 4 is connected to the power supply 44. The smoke evacuation system 100 includes the trocar 2, the smoke evacuator 1, and the suction device 3. The smoke evacuator 1 includes a controller 20 and the solenoid 13. The cautery device 4 includes the cautery instrument 41, an RF sensor 42, and a controller 43. In the present embodiment, the suction device 3, for example, always has a negative pressure and performs suction of gas.

In the cautery device 4, when the RF sensor 42 senses that the cautery instrument 41 has started a cauterizing operation, the controller 43 transmits an opening signal for opening the passage F to the smoke evacuator 1. Upon receiving the opening signal, the controller 20 of the smoke evacuator 1 controls the solenoid 13 to open the passage F. This starts the suction of the gas in the abdominal cavity of the patient 5.

When the RF sensor 42 senses that the cautery instrument 41 has stopped the cauterizing operation, the controller 43 transmits a closing signal for closing the passage F to the smoke evacuator 1. Upon receiving the closing signal, the controller 20 of the smoke evacuator 1 controls the solenoid 13 to close the passage F. This stops the suction of the gas in the abdominal cavity of the patient 5.

With the above configuration, when the smoke evacuator 1 operates in conjunction with the cautery instrument 41, smoke evacuation from the patient 5 by the smoke evacuation system 100 can be performed without contact with the smoke evacuation system 100.

### Example of Software Implementation

A control block of the surgical system 200 (particularly, the controller 20 and the controller 43) may be implemented by a logic circuit (hardware) formed in an integrated circuit (IC chip) or the like, or may be implemented by software.

In the latter case, the surgical system 200 includes a computer that executes instructions of a program that is software to implement functions. The computer includes, for example, one or more processors and a computer-readable recording medium that stores the above program. Then, in the computer, the processor reads the above program from the recording medium and executes the read program, which allows the present disclosure to be implemented. As the processor, a central processing unit (CPU) can be used, for example. As the recording medium, a “non-transitory tangible medium” such as, for example, a read only memory (ROM), a tape, a disk, a card, a semiconductor memory, a programmable logic circuit, and the like can be used. A random access memory (RAM) for loading the above program and the like may be further provided. The above program may be supplied to the computer via any transmission medium (communication network, broadcast wave, and the like) capable of transmitting the program. One aspect of the present disclosure may be implemented in the form of data signals embedded in a carrier wave in which the above program is embodied by electronic transmission.

In the present disclosure, the invention has been described above based on the various drawings and examples. However, the invention according to the present disclosure is not limited to each embodiment described above. That is, the embodiments of the invention according to the present disclosure can be varied in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

1, 1′ Smoke evacuator (gas passage adjustment mechanism)
2 Trocar (2A, 2B)
3 Suction device
4 Cautery device
5 Patient
11 Tube
12 Pressing member
13 Solenoid
14, 14′ Tube holder
15 First protruding member
16 Second protruding member (16A, 16B)
17 Holding member (17A, 17B)
18A, 18B Clamping portion
41 Cautery instrument (surgical cautery instrument)
44 Power supply
100 Smoke evacuation system
200 Surgical system

1. A gas passage adjustment mechanism comprising:
a pressing member configured to press a tube comprising a passage for gas;
a solenoid configured to move the pressing member and configured to open and close the passage through the movement;
a tube holder configured to hold the tube from a side opposite to the pressing member;
a first protruding member provided at a position facing a pressing position at which the pressing member presses the tube, the first protruding member protruding from the tube holder in a direction toward the pressing member; and
at least one second protruding member disposed at an upstream position or a downstream position of the passage with respect to the pressing position, the at least one second protruding member protruding toward a side of the tube.

2. The gas passage adjustment mechanism according to claim 1, wherein
in a cross section of the gas passage adjustment mechanism taken along a plane comprising a central axis of the tube and a central axis of the pressing member, a distance between a tip of the first protruding member and a tip of the at least one second protruding member in a direction parallel to a movement direction of the pressing member is smaller than an outer diameter of the tube in an unloaded state.

3. The gas passage adjustment mechanism according to claim 1, wherein
the tube holder comprises a pair of holding members configured to hold the tube at a predetermined position of the tube holder, each of the pair of holding members being provided at a respective one of an upstream side and a downstream side of the passage with respect to the pressing position.

4. The gas passage adjustment mechanism according to claim 1, wherein
each of a pair of the at least one second protruding member is disposed at a respective one of an upstream position and a downstream position of the passage with respect to the pressing position.

5. A gas passage adjustment mechanism comprising:
a pressing member configured to press a tube comprising a passage for gas;
a solenoid configured to move the pressing member and configured to open and close the passage through the movement;
a tube holder comprising a clamping portion configured to clamp a part of the tube from a side opposite to the pressing member; and
a first protruding member provided at a position facing a pressing position at which the pressing member presses the tube, the first protruding member protruding from the tube holder in a direction toward the pressing member.

6. The gas passage adjustment mechanism according to claim 5, wherein
the tube holder comprises a groove configured to hold the tube at a predetermined position of the tube holder.

7. The gas passage adjustment mechanism according to claim 5, wherein
the tube holder comprises a pair of the clamping portions.

8. The gas passage adjustment mechanism according to claim 1, wherein
the passage is opened at the pressing position when the solenoid is not energized, and the passage is closed at the pressing position when the solenoid is energized.

9. The gas passage adjustment mechanism according to claim 1, wherein
the solenoid is a latching solenoid.

10. The gas passage adjustment mechanism according to claim 9, wherein
the latching solenoid is configured to moves the pressing member in a pressing direction when a pulse voltage is applied, holds the pressing member by a magnetic force of a permanent magnet at a position after the movement, and move the pressing member in a direction opposite to the pressing direction when the pulse voltage is applied again.

11. The gas passage adjustment mechanism according to claim 1, wherein
a distance between inner wall surfaces of the tube is equal to or less than 3 mm at a position where the first protruding member comes into contact with the tube.

12. The gas passage adjustment mechanism according to claim 1, wherein
a distance between inner wall surfaces of the tube is equal to or less than 1.5 mm at a position where the first protruding member comes into contact with the tube.

13. A smoke evacuation system comprising:
the gas passage adjustment mechanism according to claim 1, wherein
the gas passage adjustment mechanism is configured to operate in conjunction with a surgical cautery instrument.

14. The smoke evacuation system according to claim 13, wherein
the smoke evacuation system is configured to be used in laparoscopic surgery.

15. A surgical system comprising:
the smoke evacuation system according to claim 13;
a trocar that is configured to be used in laparoscopic surgery;
the surgical cautery instrument; and
a suction device configured to suck gas in an abdominal cavity.
